(19) United States
(12) Patent Application Publication
(10) Pub. No.: US 2006/0074259 A1
(43) Pub. Date: Apr. 6, 2006

(54) Process for the enzymatic synthesis of pyroglutamic esters

(75) Inventors: Matthias Pascaly, Munster (DE); Oliver Thum, Ratingen (DE)

Correspondence Address: Leopold Presser;Scully, Scott, Murphy & Presser, 400 Garden City Plaza, Garden City, NY, 11530, US

(73) Assignee: Goldschmidt GmbH, Essen (DE)

(21) Appl. No.: 11/240,933

(22) Filed: Sep. 30, 2005

(30) Foreign Application Priority Data
Oct. 1, 2004 (DE) 10 2004 047 869.4

(51) Int. Cl. C07C 229/00

(52) U.S. Cl. 562/573

(57) ABSTRACT

A process for the preparation of compounds according to the general formula (I), 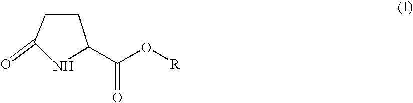 in which R is a hydrocarbon radical of an alcohol, wherein one or more alcohols of the general formula (II)
R—OH (II)
in which R is as defined above, are esterified and/or transesterified without using significant amounts of solvents, preferably without solvents, preferably under reduced pressure at temperatures below 100° C. in the presence of enzymes from the class of hydrolases with a compound according to the general formula (III), 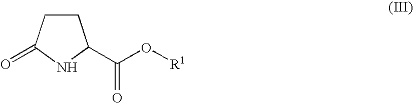
in which R¹ is either hydrogen or a linear or branched alkyl or alkenyl group having 1 to 3 carbon atoms is provided.

### FIELD OF THE INVENTION

The present invention relates to an enzymatic process for the preparation of pyroglutamic esters.

### BACKGROUND OF THE INVENTION

It is known that pyroglutamic acid (2-pyrrolidone-5-carboxylic acid) has a moisture-regulating function in that it increases the water content of the Stratum corneum of the skin even at low atmospheric humidity. The sodium salt of pyroglutamic acid is a water-soluble hygroscopic substance and a constituent of the natural moisturizing factor (NMF; Gesslein in: Conditioning Agents for Hair and Skin, page 95-110; Eds.: R. Schueller, P. Romanowski; Marcel Dekker; New York/Basel 1999). It is one of the best known moisturizers for personal care. applications.

Pyroglutamic acid or salts thereof, both of which have only limited ability to penetrate into skin and hair upon local application on account of their hydrophilicity, are rapidly washed away by water. As a consequence, long-term effects, such as continuous moisture infusion, are prevented.

For this reason, alkyl esters of pyroglutamic acid are used in cosmetic formulations in order to moisturize the skin or even to increase the rate of hair growth (see, for example, EP-B-0 342 054, EP-B-0 342 055, EP-B-0 342 056). This property is based on the ability of these compounds to penetrate through the skin into the Stratum corneum, where these compounds are cleaved by the pyroglutamate peptidase present therein (EC 3.4.19.3; J. Invest. Dermatol. 1983, 81, 122-127), releasing free pyroglutamic acid.

Frequent use in cosmetic applications has hitherto been hindered by the lack of simple and cost-effective preparation methods. The current prior art uses conventional esterification catalysts, such as, for example, sulfuric acid, thionyl chloride and hydrogen chloride, in order to obtain the desired products under extremely drastic conditions and using solvents (for example, see, DE-2102172 and DE-210217). More recently, the alternative synthesis using microwave ovens has also been described (FR-A-2 833 260).

The biocatalytic synthesis of pyroglutamic esters by enzymatic transesterification has also recently been described (Enzyme Microbial Technol. 2003, 33, 79-83; Biotechnol. Lett. 2004, 26, 193-196). However, this process also does not satisfy the requirements which are necessary for allowing industrial application.

Firstly, this prior art process is based on the use of organic solvents, which is absolutely undesired for use in cosmetic formulations and results in additional production costs.

Furthermore, even to achieve the described conversions (usually 65 to 73%) it is necessary to use large excesses of the alcohols (usually 5 equivalents) and large amounts of enzyme (up to 10% by weight).

The reaction mixture obtained in this way can then only be used for cosmetic applications following extensive work-up steps:

For example, the organic solvent and the excess alcohol have to be removed by distillation. In the case of longer-chain fatty alcohols, despite low pressures high temperatures are often still necessary, as a result of which one advantage of the enzymatic process, namely the reaction under gentle conditions, is cancelled.

Finally, this prior art process is limited to using pure ethyl or methyl pyroglutamate as the starting compound, i.e., these esters have to be synthesized in pure form, which is complex.

In view of the great potential of pyroglutamic esters and the considerable disadvantages of the production methods which are currently available, there continues to be an active requirement for a process with which these products can be prepared rapidly and cost-effectively in virtually quantitative yields starting from technical-grade low molecular weight pyroglutamic acid derivatives.

### SUMMARY OF THE INVENTION

An object of the present invention is to develop a process which satisfies precisely these requirements.

Surprisingly, it has been found that pyroglutamic acid and its esters of short-chain alcohols can be esterified or transesterified enzymatically with alcohols in a technically simple process (see Scheme 1)

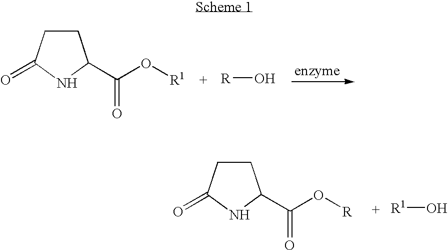

The present invention therefore provides a process for the preparation of compounds according to the general formula (I), 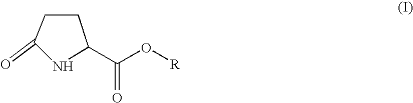
in which R is a hydrocarbon radical of an alcohol having equal to or greater than, i.e., ≧, 4 carbon atoms, wherein one or more alcohols of the general formula (II)
R—OH (II)
in which R is as defined above, are esterified and/or transesterified without using significant amounts of solvents, preferably without solvents, preferably under reduced pressure at temperatures below 100° C. in the presence of enzymes, preferably enzymes from the class of hydrolytic enzymes with a compound according to the general formula (III), 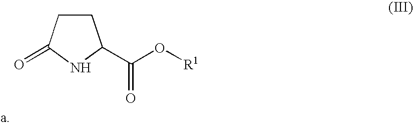
in which R¹ is either hydrogen or a linear or branched alkyl or alkenyl group having 1 to 3 carbon atoms.

The compounds of the general formula (III) used according to the invention are both pyroglutamic acid (R¹═H), its esters with short-chain alcohols where R¹=methyl, ethyl, vinyl or isopropyl radical, or technical-grade mixtures thereof, as result, for example, during an incomplete esterification of pyroglutamic acid with ethanol or methanol.

These may, for example, be mixtures of compounds in which R¹ is a hydrogen atom with those in which R¹ is a linear or branched alkyl or alkenyl group having 1 to 3 carbon atoms, in ratios in which R¹ is a hydrogen atom in an amount of from 0 to 100% by weight, preferably in an amount of from 0 to 50% by weight, in particular in an amount of from 10 to 40% by weight. Such mixtures can preferably be obtained when glutamic acid is reacted with the corresponding short-chain alcohols, preferably methanol or ethanol, under supercritical conditions, as described, for example, in the unpublished patent application DE 10 2004 008 042.9.

### DETAILED DESCRIPTION OF THE INVENTION

The present invention, which provides a process for the enzymatic synthesis of pyroglutamic esters, will now be described in greater detail. As stated above, the applicants have found that pyroglutamic acid and its esters of short-chain alcohols can be esterified or transesterified enzymatically with alcohols in a technically simple process as illustrated in Scheme 1.

The alcohols used for preparing the compounds according to the present invention are the standard commercial products having 4 to 22 carbon atoms, such as, for example, butanol, pentanol, hexanol, octanol and isomers thereof, such as isopropanol, isobutanol, 2-ethylhexanol, isononyl alcohol.

In addition, the alcohols which are prepared by known processes form monobasic fatty acids based on natural vegetable or animal oils having 6 to 22 carbon atoms, in particular having 14 to 18 carbon atoms, such as caproic acid, caprylic acid, capric acid, lauric acid, myristic acid, palmitic acid, palmitoleic acid, isostearic acid, stearic acid, 12-hydroxystearic acid, dihydroxystearic acid, oleic acid, linoleic acid, petroselic acid, elaidic acid, arachidic acid, behenic acid, erucic acid, gadoleic acid, rapeseed oil fatty acid, soya oil fatty acid, sunflower oil fatty acid, tallow oil fatty acid, palm oil fatty acid, palm kernel oil fatty acid, coconut fatty acid, which can be used on their own or in mixtures.

The ratio of the compounds of the general formula (II) and of the compounds of the general formula (III) can be varied within a wide range. This ratio should advantageously be less than 2:1, preferably less than 1.5:1, in particular about 1:1.

The enzymes which can be used according to the present invention are those from the group of hydrolytic enzymes, e.g., lipases, esterases or proteases, such as, for example, lipases from Candida rugosa, Pseudomonas sp., Thermomyces langosiosus, porcine pancreas, Mucor miehei, Alcaligines sp., cholesterol esterase from Candida rugosa, or esterase from porcine liver. Preferably, lipase B from Candida antarctica is used.

The enzymes employed in the present invention can be in free form or immobilized on suitable carriers.

According to this invention, preference is given to using immobilized lipase B from Candida antarctica in less than 5% by weight (based on the initial weight of all reactants), preferably in less than 2% by weight, as enzyme.

In the process according to the present invention, the corresponding reactants are mixed in a ratio as described above in a suitable reactor (e.g., round-bottomed flask with stirrer or in a fixed-bed reactor) and heated to the optimum working temperature of the biocatalyst used. Depending on the biocatalyst used, this temperature is from 20° C. to 100° C., preferably from 35° C. to 70° C. If a fixed-bed reactor is used, the fixed bed is charged with the selected enzyme and, after the reaction temperature has been reached, the reaction mixture is pumped through the fixed bed. When dispensing with a fixed-bed reactor, the enzyme is added directly to the reaction mixture and, when the reaction is complete, filtered off through suitable devices.

In the process according to the present invention, it is possible to dispense with the use of additional solvents of all types. To achieve the most complete conversion possible, the reaction is carried out under reduced pressure, with the resulting water of reaction (when using pyroglutamic acid), or the liberated short-chain alcohols (when using the corresponding esters) being removed by distillation. The subatmospheric pressure to be established is dependent on the reaction temperature, the boiling point of any alcohols R¹—OH to be removed, and of the reaction condensate to be removed. This pressure must ensure that the components to be removed are distilled off while as much as possible of the alcohol according to the general formula (II) used as starting material remains in the reaction vessel during the reaction.

With the process according to the present invention, quantitative conversions (determined by ¹H NMR, the detection limit of this method is a degree of conversion of about 98%) are achieved within 8 to 24 hours depending on the processing parameters (proportion of pyroglutamic acid as starting compound, type and amount of enzyme used).

The following examples are provided to illustrate the synthetic process of the present invention.

### EXAMPLE 1

### Preparation of Oleyl Pyroglutamate

In a multinecked round-bottomed flask, 19.2 g (129 mmol) of a mixture of ethyl pyroglutamate and pyroglutamic acid (molar ratio 71:29) and 34.7 g (129 mmol) of oleyl alcohol as initial charge were heated to 60° C. After adding 2.6 g of Novozym 435 (immobilized lipase B from C. antarctica), a vacuum was applied (20 mbar) and low-boiling reaction products were distilled off. The reaction was monitored by means of NMR spectroscopy or by means of a suitable chromatographic method. Conversion after 8 hours: 98%, after 24 hours: >98%. When the reaction was complete, the immobilized enzyme was filtered off. The filtrate produced 49.0 g of product (100% of the theoretical yield) without further work-up as a pale yellow liquid.

¹H NMR (DMSO-d₆, 400 MHz): δ=8.0 (br, s, 1H), 5.3 (m, 2H), 4.1 (dd, ³J=8.6 Hz, 3.9 Hz, 1H), 4.0 (td, ³J=6.6 Hz, 2.1 Hz, 2H), 2.3 (m, 1H), 2.1 (m, 2H), 2.0 (m, 5H), 1.6 (m, 2H), 1.3 (m, 22H), 0.8 (t, ³J=6.5 Hz, 3H).

### EXAMPLE 2

### Preparation of Oleyl Pyroglutamate

Analogously to Example 1, 20 g of a mixture of ethyl pyroglutamate and pyroglutamic acid (for molar ratios see Table 1) were reacted with one equivalent of oleyl alcohol and 5% by weight of Novozym 435. After 8 and 24 hours, the reaction conversion was determined by means of NMR spectroscopy (¹H NMR in DMSO-d₆).

TABLE 1
NMR spectroscopically-determined conversion of the
reaction of various mixtures of pyroglutamic acid
and ethyl pyroglutamate with oleyl alcohol.

| Acid % | Ethyl ester % | Conversion 8 h % | Conversion 24 h % |
|---|---|---|---|
| 0 | 100 | 98 | >98 |
| 25 | 75 | 98 | >98 |
| 50 | 50 | 93 | >98 |
| 75 | 25 | 91 | >98 |
| 100 | 0 | 91 | >98 |

### EXAMPLE 3

### Preparation of Oleyl Pyroglutamate

In a multinecked round-bottomed flask, 19.2 g (129 mmol) of a mixture of ethyl pyroglutamate and pyroglutamic acid (molar ratio 71:29) and 34.7 g (129 mmol) of oleyl alcohol as initial charge were heated to 60° C. After adding 1.07 g of Novozym 435 (immobilized lipase B from C. antarctica), a vacuum was applied (20 mbar) and low-boiling reaction products were distilled off. The reaction was monitored by means of NMR spectroscopy or by means of a suitable chromatographic method. Conversion after 8 hours: 95%, after 24 hours: >98%. When the reaction was complete, the immobilized enzyme was filtered off. The filtrate gave 49.0 g of product (100% of the theoretical yield) without further work-up as a pale yellow liquid.

¹H NMR (DMSO-d₆, 400 MHz): δ=8.0 (br, s, 1H), 5.3 (m, 2H), 4.1 (dd, ³J=8.6 Hz, 3.9 Hz, 1H), 4.0 (td, ³J=6.6 Hz, 2.1 Hz, 2H), 2.3 (m, 1H), 2.1 (m, 2H), 2.0 (m, 5H), 1.6 (m, 2H), 1.3 (m, 22H), 0.8 (t, ³J=6.5 Hz, 3H).

### EXAMPLE 4

### Preparation of Octyl Pyroglutamate

In a multinecked round-bottomed flask, 50.0 g (336 mmol) of a mixture of ethyl pyroglutamate and pyroglutamic acid (molar ratio 71:29) and 43.7 g (336 mmol) of octanol as initial charge were heated to 60° C. After adding 4.6 g of Novozym 435 (immobilized lipase B from C. antarctica), a vacuum was applied (20 mbar) and low-boiling reaction products were distilled off. The reaction was monitored by means of NMR spectroscopy or by means of a suitable chromatographic method. Conversion after 8 hours: 95%, after 24 hours: >98%. When the reaction was complete, the immobilized enzyme was filtered off. The filtrate gave 81 g of product (100% of the theoretical yield) without further work-up as a pale yellow liquid.

¹H NMR (DMSO-d₆, 400 MHz): δ=8.0 (br, s, 1H), 4.2 (dd, ³J=8.6 Hz, 4.3 Hz, 1H), 4.1 (t, ³J=6.5 Hz, 2H), 2.3 (m, 1H), 2.1 (m, 2H), 2.0 (m, 1H), 1.6 (m, 2H), 1.3 (m, 10H), 0.8 (t, ³J=7.1 Hz, 3H).

### EXAMPLE 5

### Preparation of Lauryl Pyroglutamate

In a multinecked round-bottomed flask, 51.0 g (339 mmol) of a mixture of ethyl pyroglutamate and pyroglutamic acid (molar ratio 77:23) and 63.1 g (339 mmol) of lauryl alcohol as initial charge were heated to 60° C. After adding 5.7 g of Novozym 435 (immobilized lipase B from C. antarctica), a vacuum was applied (20 mbar) and low-boiling reaction products were distilled off. The reaction was monitored by means of NMR spectroscopy or by means of a suitable chromatographic method. Conversion after 8 hours: 97%, after 24 hours: >98%. When the reaction was complete, the immobilized enzyme was filtered off. The filtrate gave 100.7 g of product (100% of the theoretical yield) without further work-up as a pale yellowish solid.

¹H NMR (DMSO-d₆, 400 MHz): δ=8.0 (br, s, 1H), 4.2 (dd, ³J=8.6 Hz, 3.5 Hz, 1H), 4.1 (t, ³J=6.5 Hz, 2H), 2.3 (m, 1H), 2.1 (m, 2H), 2.0 (m, 1H), 1.6 (m, 2H), 1.3 (m, 18H), 0.8 (t, ³J=7.1 Hz, 3H).

### EXAMPLE 6

### Preparation of 2-hexyldecyl Pyroglutamate

In a multinecked round-bottomed flask, 27.3 g (177 mmol) of a mixture of ethyl pyroglutamate and pyroglutamic acid (molar ratio 90:10) and 42.9 g (177 mmol) of 2-hexyldecyl alcohol (Isofol-16) as initial charge were heated to 60° C. After adding 3.4 g of Novozym 435 (immobilized lipase B from C. antarctica), a vacuum is applied (20 mbar) and low-boiling reaction products were distilled off. The reaction was monitored by means of NMR spectroscopy or by means of a suitable chromatographic method. Conversion after 8 hours: 97%, after 24 hours: >98%. When the reaction was complete, the immobilized enzyme was filtered off. The filtrate gave 62.5 g of product (100% of the theoretical yield) without further work-up as a pale yellowish solid.

¹H NMR (DMSO-d₆, 400 MHz): δ=8.0 (br, s, 1H), 4.2 (m, 1H), 4.1 (m, 2H), 2.3 (m, 1H), 2.1 (m, 2H), 1.9 (m, 1H), 1.6 (m, 2H), 1.3 (m, 24H), 0.8 (t, ³J=7.1 Hz, 6H).

The above examples and embodiments are given to illustrate the scope and spirit of the present invention. These examples and embodiments will make apparent, to those skilled in the art, other examples and embodiments. Those other examples and embodiments are within the contemplation of the present invention. Therefore, the present invention should be limited only by appended claims.

What is claimed is:

1. A process for the preparation of compounds according to the general formula (I), 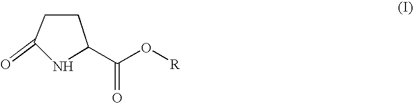
in which R is a hydrocarbon radical of an alcohol having ≧4 carbon atoms, wherein one or more alcohols of the general formula (II)
R—OH (II)
in which R is as defined above, are esterified and/or transesterified in the presence of enzymes with a compound according to the general formula (III), 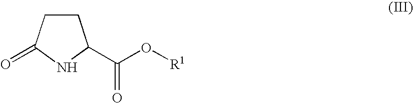
in which R¹ is either hydrogen or a linear or branched alkyl or alkenyl group having 1 to 3 carbon atoms.

2. The process of claim 1, wherein, in the alcohols of the general formula (II), R is an optionally branched hydrocarbon radical having 4 to 22 carbon atoms which optionally contains multiple bonds.

3. The process of claim 1, wherein a ratio of the compounds of the general formula (II) to the compounds of the general formula (III) of less than 2:1 is employed.

4. The process of claim 3, wherein the ratio of compounds of the general formula (II) to the compounds of the general formula (III) is less than 1.5:1.

5. The process of claim 4, wherein the ratio of compounds of the general formula (II) to the compounds of the general formula (III) is about 1:1.

6. The process of claim 1, wherein mixtures of compounds of the general formula (III) where R¹═H and R¹=C₁₋₃-alkyl in ratios in which R¹ is a hydrogen atom in an amount of from 0 to 100% by weight are used.

7. The process of claim 6, wherein said amount is from 0 to 50% by weight.

8. The process of claim 6, wherein said amount is from 10 to 40% by weight.

9. The process of claim 6, in which the compounds of the general formula (III) used are mixtures of compounds in which R¹ is a hydrogen atom with those in which R¹ is a linear or branched alkyl or alkenyl group having 1 to 3 carbon atoms which have come from the reaction of glutamic acid with the corresponding short-chain alcohol under supercritical conditions.

10. The process of claim 1, wherein an immobilized enzyme is used.

11. The process of claim 10, wherein said immobilized enzyme is a lipase, an esterase or a protease.

12. The process of claim 10, wherein said immobilized enzyme is lipase B from Candida Antarctica.

13. The process of claim 1, wherein said esterified and/or transesterified is performed under reduced pressure and at a temperature below 100° C.
